(19) United States
(12) Patent Application Publication
(10) Pub. No.: US 2020/0078484 A1
(43) Pub. Date: Mar. 12, 2020

(54) SYSTEM AND METHOD FOR HYDROGEN PEROXIDE DISINFECTION COMPLIANCE MONITORING

(71) Applicant: Microban Products Company, Huntersville, NC (US)

(72) Inventors: Mai Le Phuong Ha, Cornelius, NC (US); Karen Terry Welch, Kannapolis, NC (US); Bruno Michael Mourao, Cornelius, NC (US); Gina Parise Sloan, Statesville, NC (US)

(21) Appl. No.: 16/562,256

(22) Filed: Sep. 5, 2019

Related U.S. Application Data
(60) Provisional application No. 62/728,511, filed on Sep. 7, 2018.

(51) Int. Cl. A61L 2/28; A61L 2/20; A61L 12/12

(52) U.S. Cl. CPC A61L 2/28; A61L 12/128; A61L 2/208

(57) ABSTRACT

The present invention relates to a system and a method of using the system to detect any dried phosphates on a surface, more particularly to a system and a method of using the system for disinfection compliance monitoring of hydrogen peroxide. The system may be in a form of a detection kit.

### CROSS-REFERENCE TO RELATED APPLICATION

This application claims priority from U.S. Provisional Patent Application No. 62/728,511, filed on Sep. 7, 2018, in the United States Patent and Trademark Office. The disclosure of which is incorporated herein by reference in its entirety.

### FIELD OF THE INVENTION

The present invention relates to a system and method for disinfection compliance monitoring, more particularly to a system and method for hydrogen peroxide disinfection compliance monitoring.

### BACKGROUND OF THE INVENTION

At present, hydrogen peroxide is the only active on the Food and Drug Administration (FDA) Generally Recognized as Safe (GRAS) list and has seen its use gradually increase. This chemical is employed in consumer, professional, food service and healthcare applications. However, there are limited means to determine if hydrogen peroxide was utilized to clean a surface, if the chemistry adequately covered all target surfaces, or if there is any residual chemistry providing extended protection. There is a need for a compliance monitoring tool to ensure that a surface was cleaned with a disinfectant and that the correct chemistry was utilized to ensure end user safety and material longevity. There are known techniques that allow one to clearly see if a liquid is on the surface at the time of application or a within a short period thereafter. However, these approaches do not identify whether the correct disinfection chemistry was applied to the surface, only that the appropriate contact time was utilized.

Hydrogen peroxide is highly unstable. It decomposes into oxygen and water, leaving no residual. To stabilize hydrogen peroxide during normal storage and handling conditions, phosphate-based substances such as sodium pyrophosphate, organophosphate or alternative phosphate based chemistries are commonly used. After hydrogen peroxide decomposes into oxygen and water, the stabilizers remain on the surface. Some phosphate detection assays take hours to complete.

Thus, there is a need to have a hydrogen peroxide detection kit or system allowing the measurement of disinfection and sanitization chemistries post application to a surface and without surface damage or alteration. More particularly, there is not a method or system to detect dried phosphates.

### SUMMARY OF THE INVENTION

The present invention relates to a system and a method of using the system to detect any dried phosphates on a surface, more particularly to a system and a method of using the system for disinfection compliance monitoring of hydrogen peroxide. The system may be in a form of a detection kit used for the measurement of disinfection and sanitization chemistries post application to a surface without surface damage or alteration. The system of the present invention allows for immediate naked eye detection.

In an embodiment of the invention, a detection system for detection of a dried residual phosphate compound on a surface is provided. The detection system comprises a dry state part comprising a metal indicator dye, and a wet state part comprising water, wherein 2,6-Bis(bis(2-pyridylmethyl)aminomethyl)-4-methylphenol, a water-soluble salt of a transition metal having a di- or trivalent charge, or a combination thereof is present in either the wet state part or the dry state part.

In an embodiment of the invention, a detection, kit is provided comprising an indicator composition. The indicator composition comprises a metal indicator dye, a water-soluble salt of a transition metal having a di- or trivalent charge, and 2,6-Bis(bis(2-pyridylmethyl)aminomethyl)-4-methylphenol (H-bpmp).

In an embodiment of the invention, a method of using a detection system having at least two parts comprises combining a dry state part comprising a metal indicator dye with a wet state part comprising water to form the detection system, wherein H-bpmp, zinc perchlorate, or a combination thereof are present in either the wet state part or the dry state part, and detecting a dried residual phosphate compound on a surface with the detection system.

Further areas of applicability of the present invention will become apparent from the detailed description provided hereinafter. It should be understood that the detailed description and specific examples, while indicating the preferred embodiments of the invention, are intended for purposes of illustration only and are not intended to limit the scope of the invention.

### DESCRIPTION OF THE PREFERRED EMBODIMENTS

The following description of the embodiments of the present invention is merely exemplary in nature and is in no way intended to limit the invention, its application, or uses. The present invention has broad potential application and utility, which is contemplated to be adaptable across a wide range of industries. The following description is provided herein solely by way of example for purposes of providing an enabling disclosure of the invention, but does not limit the scope or substance of the invention.

In an embodiment of the invention, a method of using a chemical composition for detection of a residual phosphate compound on a surface is provided. The method of the present invention detects a dried residual phosphate compound on a surface.

In an embodiment of the invention, a method of using a chemical composition for detection of dried hydrogen peroxide on a surface is provided.

In an embodiment of the invention, a detection kit for detection of application of hydrogen peroxide to a surface is provided. The detection kit comprises an indicator composition.

The indicator composition of the present invention preferably comprises a metal indicator dye, a water-soluble salt of a transition metal capable of having a di- or trivalent charge, and 2,6-Bis(bis(2-pyridylmethyl)aminomethyl)-4-methylphenol (H-bpmp).

The metal indicator dye may be in a range of about 0.025 mM to about 1 mM. Non-limiting examples of the metal indicator dye include, but are not limited to, Pyrocatechol violet, Xylenol Red, and combinations thereof.

The water-soluble salt of a transition metal capable of having a di- or trivalent charge may be in a range of about 0.25 mM to about 50 nM. Non-limiting examples of the water-soluble salt include, but are not limited to, Zinc Perchlorate, Iron Perchlorate, and combinations thereof.

The b2,6-Bis(bis(2-pyridylmethyl)aminomethyl)-4-methylphenol (H-bpmp) may be in a range of about 0.025 mM to about 1 mM.

In an embodiment of the invention, a detection system of the present invention overcomes a stability issue with the chemistry by separating out the components, thereby allowing for a long shelf life and a subsequent commercially viable solution. The detection system includes at least two separated parts, wherein at least one part is in a dry state and a second part is in a wet state. The detection system may be in a form of a detection kit.

The detection system comprises: a dry state part that includes the metal indicator dye, and a wet state part that includes water, preferably having a pH greater than or equal to 7.5. H-bpmp, zinc perchlorate, or a combination thereof are present in either the wet state part or the dry state part. The dry state part can be held on or in a substrate. The wet state part is to be released at point of use so the chemicals in the wet state and dry state parts can react with each other. The presence of any dried residual phosphate compounds on a surface is determined by the detection system.

In an embodiment of the invention, a method of using the detection system having at least two parts comprises combining a dry state part comprising a metal indicator dye with a wet state part comprising water, and detecting any dried residual phosphate compounds on a surface using the detection system. H-bpmp, zinc perchlorate, or a combination thereof are present in either the wet state part or the dry state part.

### EXAMPLE

In an example, pyrocatechol violet, H-bpmp and zinc perchlorate were incorporated and dried onto a cotton swab. The swab was a regular swab and was originally white in color. The dried swab was brownish in color. Right before use, high pH water was used to wet the swab. The swab turned blue upon contact with the high pH water. The swab color did not change when it was used to swab a surface previously cleaned with quaternary ammonium or bleach-based disinfectants. The blue color on the swab changed to bright yellow when swabbed on a surface cleaned with hydrogen peroxide.

### Comparative Example

A solution was prepared in accordance with Han et. al. (Han and Kim 2002). The Han solution contained 50 μM H-bpdmp, 100 μM zinc perchlorate, and 50 μM pyrocatechol violet. The Han solution was light blue in color and changed to clear, yellow when phosphate was introduced. A color change was, described as occurring between a pH of 6.5 to 7.5 and most prominent at a pH of 7. The solution as indicated by Han et. al. was not stable for more than a day. Precipitation within the liquid itself was observed within 24 hours therefore rendering the liquid unusable for commercialization. Additionally, complete color loss was noted if the solution was dried on an applicator substrate, once again rendering the formula as disclosed by Han et. al. to be non-commercially viable. Since shelf life stability is a critical factor in creating a viable commercial product, the formulation as described by Han et. al. could not be utilized for the purposes of compliance monitoring. Unlike Han et al., in the present invention the phosphates were not in solution, the concentration of the components was increased in a non-ratiometric manner, pH was altered, and the components were separated to allow for stability.

It will therefore be readily understood by those persons skilled in the art that the present invention is susceptible of broad utility and application. Many embodiments and adaptations of the present invention other than those herein described, as well as many variations, modifications and equivalent arrangements, will be apparent from or reasonably suggested by the present invention and the foregoing description thereof, without departing from the substance or scope of the present invention. Accordingly, while the present invention has been, described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements.

What is claimed is:

1. A detection system comprising:
a dry state part comprising a metal indicator dye, and
a wet state part comprising water,
wherein 2,6-Bis(bis(2-pyridylmethyl)aminomethyl)-4-methylphenol, a water-soluble salt of a transition metal having a di- or trivalent charge, or a combination thereof is present in either the wet state part or the dry state part, the detection system being for detection of a dried residual phosphate compound on a surface.

2. A detection kit comprising:
an indicator composition, wherein the indicator composition comprises a metal indicator dye, a water-soluble salt of a transition metal having a di- or trivalent charge, and 2,6-Bis(bis(2-pyridylmethyl)aminomethyl)-4-methylphenol (H-bpmp).

3. The detection kit according to claim 2, wherein the metal indicator dye is in a range of about 0.025 mM to about 1 mM.

4. The detection kit according to claim 2, wherein the metal indicator dye is selected from the group consisting of Pyrocatechol violet, Xylenol Red, and a combination thereof.

5. The detection kit according to claim 2, wherein the water-soluble salt of the transition metal having a di- or trivalent is in a range of about 0.25 mM to about 50 mM.

6. The detection kit according to claim 2, wherein the water-soluble salt is selected from the group consisting of zinc perchlorate, iron perchlorate, and a combination thereof.

7. The detection kit according to claim 2, wherein the 2,6-Bis(bis(2-pyridylmethyl)aminomethyl)-4-methylphenol (H-bpmp) is in a range of about 0 025 mM to about 1 nM.

8. The detection kit according to claim 2, wherein the detection kit is for hydrogen peroxide detection.

9. A method of using a detection system having at least two parts, the method comprising:
combining a dry state part comprising a metal indicator dye with a wet state part comprising water to form the detection system, wherein H-bpmp, zinc perchlorate, or a combination thereof are present in either the wet state part or the dry state part, and
detecting a dried residual phosphate compound on a surface with the detection system.

10. The method according to claim 9, wherein a color change is exhibited upon detection.

11. The method according to claim 9, wherein the dried residual phosphate compound is from hydrogen peroxide.
